SEQUENCE LISTING (1 sequence)

SEQ ID NO: 1 - protein, 905 aa, Synthetic construct
HGEGTFTSDLSKQMEEEAVRLFIEWLKNGGPSSGAPPPSGGSPAGSPTSTEEGTSESATPESGPGTSTEPSEGSAPGSPAGSPTSTEEGTSTEPSEGSAPGTSTEPSEGSAPGTSESATPESGPGSEPATSGSETPGSEPATSGSETPGSPAGSPTSTEEGTSESATPESGPGTSTEPSEGSAPGTSTEPSEGSAPGSPAGSPTSTEEGTSTEPSEGSAPGTSTEPSEGSAPGTSESATPESGPGTSTEPSEGSAPGTSESATPESGPGSEPATSGSETPGTSTEPSEGSAPGTSTEPSEGSAPGTSESATPESGPGTSESATPESGPGSPAGSPTSTEEGTSESATPESGPGSEPATSGSETPGTSESATPESGPGTSTEPSEGSAPGTSTEPSEGSAPGTSTEPSEGSAPGTSTEPSEGSAPGTSTEPSEGSAPGTSTEPSEGSAPGSPAGSPTSTEEGTSTEPSEGSAPGTSESATPESGPGSEPATSGSETPGTSESATPESGPGSEPATSGSETPGTSESATPESGPGTSTEPSEGSAPGTSESATPESGPGSPAGSPTSTEEGSPAGSPTSTEEGSPAGSPTSTEEGTSESATPESGPGTSTEPSEGSAPGTSESATPESGPGSEPATSGSETPGTSESATPESGPGSEPATSGSETPGTSESATPESGPGTSTEPSEGSAPGSPAGSPTSTEEGTSESATPESGPGSEPATSGSETPGTSESATPESGPGSPAGSPTSTEEGSPAGSPTSTEEGTSTEPSEGSAPGTSESATPESGPGTSESATPESGPGTSESATPESGPGSEPATSGSETPGSEPATSGSETPGSPAGSPTSTEEGTSTEPSEGSAPGTSTEPSEGSAPGSEPATSGSETPGTSESATPESGPGTSTEPSEGSAPG

(19) United States
(12) Patent Application Publication
(10) Pub. No.: US 2023/0331859 A1
(43) Pub. Date: Oct. 19, 2023

(54) METHODS FOR TREATING SHORT BOWEL SYNDROME AND/OR HIGH OUTPUT OSTOMY

(71) Applicant: 9 Meters Biopharma, Inc., Raleigh, NC (US)

(72) Inventors: Patrick H. GRIFFIN, Raleigh, NC (US); Nir BARAK, Tel-Aviv (IL); Sireesh APPAJOSYULA, Raleigh, NC (US)

(21) Appl. No.: 18/310,796

(22) Filed: May 2, 2023

Related U.S. Application Data
(63) Continuation-in-part of application No. PCT/US21/58522, filed on Nov. 9, 2021.
(60) Provisional application No. 63/111,869, filed on Nov. 10, 2020.

(51) Int. Cl. C07K 16/28; A61P 1/00; A61K 9/00

(52) U.S. Cl. CPC C07K 16/2869; A61P 1/00; A61K 9/0019; A61K 38/00

(57) ABSTRACT

The present invention provides methods for treating short bowel syndrome and/or high output ostomy. The invention comprises administering to a patient in need a long-acting GLP-1 receptor agonist. In various embodiments, the method reduces ostomy output, reduces requirement for parenteral nutrition, and/or ameliorates acute intestinal failure.

### CROSS-REFERENCE TO RELATED APPLICATIONS

This application is a continuation in part of PCT Application No. PCT/US21/58522, filed Nov. 09, 2021, which claims priority to, and the benefit of, U.S. Provisional Application No. 63/111,869, filed Nov. 10, 2020, each of which is hereby incorporated by reference in their entirety.

### DESCRIPTION OF THE SEQUENCE LISTING SUBMITTED ELECTRONICALLY

This application contains a Sequence Listing in XML format submitted electronically herewith via Patent Center. The contents of the XML file submitted electronically herewith are incorporated herein by reference in their entirety: A XML copy of the Sequence Listing (Filename: “NMT-027CP_116031-5030.xml”; Date created: May 2, 2023; File size: 2,821 bytes).

### BACKGROUND

Ostomy surgery is a procedure that allows bodily waste to pass through a surgically created stoma into a prosthetic, such as an ostomy bag on the outside of the body. An ostomy may be necessary in the case of birth defects, cancer, inflammatory bowel disease (e.g., Crohn's disease or ulcerative colitis), or severe abdominal or pelvic trauma, for example. In some cases, the patient has a surgical removal of a large portion of the small intestine (e.g., due to Crohn's disease, or necrotizing enterocolitis in the case of young children) leading to short bowel syndrome (SBS), a malabsorption disorder caused by a lack of functional small intestine.

Patients with a jejunostomy (i.e., end jejunostomy) have major problems of dehydration, electrolyte depletion, and nutritional challenges, largely due to a large volume of stomal output. Some SBS patients fail to adapt adequately and have high ostomy outputs from the time of surgery, and may experience intestinal failure. Intestinal failure generally requires i.v. fluid and parenteral nutritional support. Improved treatment of SBS as well as conditions requiring ostomy surgery are critical for reversing or preventing intestinal failure and/or ameliorating the need for parenteral nutritional support and/or frequent i.v. fluids. In the various embodiments, this disclosure meets these objectives.

### BRIEF DESCRIPTION OF THE DRAWINGS

FIG. 1 is a graphical representation of the mean 24-hour total stool output (TSO) during a 6-week observational period for a 4-week dosing regimen of the long-lasting GLP-1 agonist (SEQ ID NO: 1), shown as percent change from baseline (n=11).

FIG. 2 is a graphical representation of the sustained total stool output (TSO) reduction during a 6-week observational period for a 4-week dosing regimen of the long-lasting GLP-1 agonist (SEQ ID NO: 1), shown as percent change from baseline (CFB).

FIG. 3 is a graphical representation of the pharmacokinetic (PK) profile observed during a 4-week dosing regimen of the long-lasting GLP-1 agonist (SEQ ID NO: 1).

### DETAILED DESCRIPTION

The present invention provides methods for treating short bowel syndrome and/or high output ostomy. The invention comprises administering to a patient in need a long-acting Glucagon-like peptide 1 (GLP-1) receptor agonist. Thus, the disclosure provides for a use of a long acting GLP-1 receptor agonist for treating short bowel syndrome and/or high output ostomy. In embodiments, the long-acting Glucagon-like peptide 1 (GLP-1) receptor agonist is the exenatide-XTEN fusion protein known as vurolenatide. In various embodiments, the method reduces ostomy output, reduces requirement for parenteral nutrition and/or frequent i.v. fluid support, and/or ameliorates acute intestinal failure. In some embodiments, the method of the present invention reduces high urine and/or stool output in a patient having short bowel syndrome, including end jejunostomy patients.

In some embodiments, the patient is an end jejunostomy patient experiencing a high output ostomy (HOO). HOO is defined herein as more than about 1.5 L of fluid from the ostomy in a 24-hour period. In some embodiments, the patient is losing at least about 2 L of fluid from the ostomy in a 24-hour period, or at least about 2.5 L, or at least about 3 L, or at least about 3.5 L, or at least about 4 L of fluid from the ostomy in a 24-hour period.

Under normal circumstances, 9 to 10 liters of fluid enter the jejunum each day, including both oral intake and gastrointestinal secretions from oral, gastric, duodenal, and biliopancreatic sources. The jejunum absorbs approximately 6 L, and the ileum another 2.5 liters, leaving approximately 1 to 1.5 liters of fluid entering the colon per day. Almost all of this fluid and the electrolytes it contains are absorbed in the colon, leaving approximately 100 milliliters excreted in feces daily. Thus, diversion of the fecal stream at the level of the ileocecal valve would be expected to produce approximately 1 to 1.5 liters of stool output per day containing approximately 200 mEq of sodium, 100 mEq of chloride, and 10 mEq of potassium. Diversion at a higher level will result in significantly more fluid and electrolyte losses.

Fluid and electrolyte losses can be offset in part by increased oral intake. In addition, in the case of SBS, the remaining small intestine often compensates by increasing the efficiency of fluid and electrolyte absorption, a process termed adaptation, which occurs within weeks to months. This adaptation process is a combination of: (1) changes in mucosal morphology—mucosal hypertrophy and hyperplasia of remaining intestines that increases its absorptive capacity; (2) changes in small bowel electrolyte transport; and (3) alterations in motility—slowing transit through the bowel increases time available for absorption (Rowe, K. et al., Proc (Bayl. Univ. Med. Cent.), 33(2):218-226, 2020).

In various embodiments, the patient has Short Bowel Syndrome (SBS). In some embodiments, the patient having SBS has high output of urine and/or stool. SBS is a life-altering condition caused by a significant shortening of the gastrointestinal (GI) tract, often from surgical removal of a large portion of the small intestine. In some embodiments, a patient having SBS has an intact colon (i.e., where the remaining ends of the intestine are stitched together). In other embodiments, a patient having SBS does not have an intact colon, and the patient relies on a permanent ostomy. If the shortened GI tract cannot properly absorb nutrients, the patient may need to rely on total parenteral nutrition (TPN) to manage nutritional needs. TPN involves intravenous infusion for up to several hours every day which, aside from the negative impact on quality of life, carries side effects as well as high risk of systemic infections. Further, high output ostomy can be a severe and chronic result of short bowel syndrome. In some embodiments, the patient has an end jejunostomy. In various embodiments, the patient to be treated does not have active Crohn' s disease or IBD. For example, in various embodiments, if the patient has a history of Crohn's disease or IBD, the patient is in remission.

Patients with SBS and HOO are at risk for intestinal failure. Three types of intestinal failure can be distinguished. Type 1 acute intestinal failure (IF) is self-limiting and common following abdominal surgery, and the necessity of i.v. fluid and nutritional support is short (<28 days). Type 2 IF is also acute in onset and reversible, but requires more than 28 days of intravenous fluid and/or nutritional support and is often associated with septic, metabolic and complex nutritional complications. Finally, type 3 IF is characterized as chronic IF, frequently irreversible and requires long term or even lifelong therapy with intravenous fluid and/or TPN. In various embodiments, the subject may be experiencing or at risk of Type 1, Type 2, or Type 3 Intestinal Failure.

In accordance with this disclosure, the patient's condition is ameliorated by administration of a long-acting GLP-1 receptor agonist. Glucagon-like peptide-1 (GLP-1) is a hormone that is produced by L-cells in the ileum. As an incretin hormone, GLP-1 stimulates insulin secretion in a glucose-dependent process, and it tonically suppresses glucagon release and lowers postprandial glucagon excursions (Schirra, J., et al., Gut; 55:243-251, 2006). GLP-1 is also a regulator of fasting and postprandial gastroduodenal motility. GLP-1 also regulates several GI functions, such as decreasing gastric acid secretion, and slowing GI transit and motility. GLP-1 (e.g., GLP-1 (7-37)) and exenatide are peptide GLP-1 receptor agonists that can be delivered for pharmaceutical intervention. In various embodiments, the GLP-1 receptor agonist may have from one to five, or from one to three amino acid substitutions with respect to human GLP-1 peptide or exenatide.

In accordance with embodiments of this disclosure, the GLP-1 receptor agonist is a long-acting GLP-1 receptor agonist unit dose providing an effective concentration of the GLP-1 receptor agonist in circulation for at least about one week or at least about two weeks upon a single subcutaneous injection. In some embodiments, the long-acting GLP-1 receptor agonist formulation is vurolenatide. In accordance with this disclosure, a large AUC of GLP-1 agonist (e.g., as achieved herein by a long-acting GLP-1 agonist such as vurolenatide) is believed to be superior for treatment of SBS or HOO as compared to providing spikes (e.g., one or more times daily) in levels of GLP-1 receptor agonist.

The long-acting GLP-1 receptor agonist may be created using various chemical polymers, which can be conjugated to the GLP-1 receptor agonist to enhance the pharmacokinetic (PK) properties. Exemplary PK properties include longer terminal half-life, larger area under the curve, increased time in which the blood concentration remains within the therapeutic window, increased time between consecutive doses, and decreased dose in moles over time. In some embodiments, the GLP-1 agonist is conjugated or fused to a chemical polymer, such as polyethylene glycol (PEG), polylactides (PLA), polyglycolides (PGA), poly(lactide-co-glycolide) (PLGA), poly(ϵ-caprolactone) (PCL), polyglyconate, polyanhydrides, polyorthoesters, poly(dioxanone), and polyalkylcyanoacrylates. In some embodiments, the GLP-1 receptor agonist is acylated.

In other embodiments, the GLP-1 receptor agonist is fused to a half-life extension polypeptide, such as an albumin amino acid sequence, a transferrin amino acid sequence, an immunoglobulin Fc domain amino acid sequence (e.g., IgG1 or IgG4 Fc domain), an elastin-like peptide amino acid sequence, or an unstructured polypeptide as disclosed in U.S. Pat. No. 8,957,021, which is hereby incorporated by reference. An exemplary unstructured polypeptide is AE864XTEN, and is exemplified by the fusion protein of SEQ ID NO: 1 (vurolenatide). Other half-life extension polypeptides are disclosed in U.S. Pat. Nos. 8,748,380, 10,233,228, 10,501,524, and 9,458,218, which are hereby incorporated by reference in its entirety. Exemplary fusions may include linker sequences, such as sequences of from 4 to 40 amino acids (e.g. 4 to 20), and which are optionally composed predominately of serine and glycine amino acids.

In some embodiments, the long-acting GLP-1 receptor agonist is an exenatide or GLP-1 peptide fusion with a half-life extension polypeptide at the C-terminus. In some embodiments, the long-acting GLP-1 receptor agonist comprises or consists of the amino acid sequence of SEQ ID NO: 1.

In some embodiments, the long-acting GLP-1 receptor agonist (such as the long-acting GLP-1 receptor agonist of SEQ ID NO: 1) is administered at a unit dose of about 12 mg to about 250 mg, or a unit dose of about 25 mg to about 200 mg. For example, the unit dose may be about 50 mg to about 150 mg. Exemplary unit doses include about 25 mg, about 50 mg, about 75 mg, about 100 mg, about 125 mg, about 150 mg, about 175 mg, and about 200 mg. The route of administration of the long-acting GLP-1 receptor agonist is by parenteral administration, such as subcutaneous injection. In some embodiments, the long-acting GLP-1 receptor agonist is administered by intravenous injection.

In some embodiments, the long-lasting GLP-1 receptor agonist (e.g., SEQ ID NO: 1) is administered as a 50 mg unit dose, subcutaneously, once every 2 weeks (i.e. 50 mg

Q2W). In some embodiments, 50 mg Q2W dosing can result in an average daily reduction in ostomy output of at least about 10%, or at least about 15%, or at least about 20%, or at least about 25%, or at least about 30%, or at least about 35%, or at least about 40% or at least about 45%, or at least about 50% from patient baseline. In some embodiments, 50 mg Q2W dosing can result in a reduction of parenteral support by at least about 10%, or at least about 15%, or at least about 20%, or at least about 25%, or at least about 30%, or at least about 35%, or at least about 40% or at least about 45%, or at least about 50% from patient baseline. This treatment regimen can be continued as the patient exhibits HOO, or as needed to reduce reliance on TPN or frequent i.v. fluid support. In some embodiments, the patient receives at least 2 doses of the long-lasting GLP-1 receptor agonist, or at least 3, or at least 4, or at least 5, 6, 8, 10, 12, or more doses. In some embodiments, the treatment is continuous and can be life-long treatment.

In some embodiments, the long-acting GLP-1 receptor agonist is administered about once weekly, or about twice monthly, or about once monthly. The treatment regimen may be continued during the time that the patient exhibits HOO, or as needed to reduce reliance on TPN or frequent i.v. fluid support. In various embodiments, the patient is administered at least two doses of the long-acting GLP-1 agonist. In some embodiments, the patient is administered at least three, or at least four, or at least five, or at least six doses of the long-acting GLP-1 receptor agonist. In some embodiments, the subject receives about 10 doses of the long-acting GLP-1 receptor agonist or less, or receives about 8 doses of the long-acting GLP-1 receptor agonist, or less. In still other embodiments, therapy with the long-acting GLP-1 receptor agonist is continuous. In some embodiments, the subject receives life-long administration of the long-acting GLP-1 receptor agonist. In some embodiments, during therapy, the patient exhibits an average daily reduction of ostomy output of at least about 10%, or at least about 15%, or at least about 20%, or at least about 25%, or at least about 30%, or at least about 35%, or at least about 40%, or at least about 50% from baseline. In some embodiments, during treatment, the patient's parenteral support is reduced by at least about 10%, or at least about 15%, or at least about 20%, or at least about 25%, or at least about 30%, or at least about 35%, or at least about 40%, or at least about 50% from baseline. In various embodiments, significant improvement from baseline is observed in the first 24 hours or first 48 hours of treatment. In various embodiments, the long-acting GLP-1 receptor agonist therapy described herein provides improvement in bowel movement frequency and form, for example, bowel movements become no longer substantially meal-related. In some embodiments, the long-acting GLP-1 receptor agonist therapy described herein reduces diarrhea occurrences, for example, reduces, eliminates or avoids chronic unrelenting diarrhea. These improvements in some embodiments occur after the first dose of the long-acting GLP-1 receptor agonist. Accordingly, the therapy described herein can substantially improve bowel habits, nutritional status, and quality of life for SBS patients.

In some aspects and embodiments, this disclosure provides a method for treating a patient having short bowel syndrome. The method comprises administering to the patient by subcutaneous injection a unit dose (as described herein) of the long-acting GLP-1 receptor agonist of SEQ ID NO: 1. In various embodiments, the administration is from about once weekly to about once monthly. For example, the patient having SBS may have an end jejunostomy, and the patient may be experiencing high output ostomy (as described above). In exemplary embodiments, the unit dose of the long-acting GLP-1 agonist is about 50 mg to about 150 mg. For example, the unit dose may be about 50 mg, about 100 mg, or about 150 mg. In some embodiments, the long-acting GLP-1 receptor agonist is administered about twice weekly, about once weekly, or about twice monthly, or about once monthly. In some embodiments, about 50 mg, or about 100 mg, or about 150 mg are administered once or twice weekly. In some embodiments, about 50 mg, or about 100 mg, or about 150 mg are administered once or twice monthly. Thus, in exemplary embodiments, about 50 mg, or about 100 mg, or about 150 mg are administered about twice monthly. In some embodiments, about 150 mg of the GLP-1 receptor agonist is administered about monthly.

In various embodiments, the patient does not experience substantial adverse effects from the drug, such as substantial symptoms of nausea, vomiting, hypoglycemia, pancreatitis, liver injury, and/or kidney function. In various embodiments, the adverse effects (such as nausea and vomiting) are significantly less than observed with agents such as liraglutide or exenatide.

In some embodiments, the administration (e.g., 50 mg Q2W) does not result in substantial stacking effects. A stacking effect, as referred to in embodiments herein, is the undesirable accumulation of a drug. With regard to pharmacokinetics, stacking effects can be evident with long-lasting drugs intended for repeated dosing.

In some embodiments, the unit dose is suspended in an appropriate physiological solution, e.g., saline or other pharmacologically acceptable solvent or a buffered solution, and may optionally comprise a surfactant. Pharmaceutically acceptable carriers include water, saline, and glycerol. In some embodiments, the formulation may comprise fixed oils, polyethylene glycol, propylene glycol or other solvents. In some embodiments, the solvent is water.

In various embodiments, the unit dose can be delivered using a syringe, injection pen, or autoinjector for convenient self-administration. Auto-injectors such as an “injection pen” are spring-loaded syringes designed to deliver a dose of a particular drug. By design, injection pens are easy to use and are intended for self-administration by patients, or administration by untrained personnel. Injection pens are designed to overcome the hesitation associated with self-administration of the needle-based drug delivery device. The injection pen keeps the needle tip shielded prior to injection and also has a passive safety mechanism to prevent accidental firing (injection). Injection depth can be adjustable or fixed and a function for needle shield removal may be incorporated. By pressing a button, the syringe needle is automatically inserted into the subcutaneous tissue and the drug is delivered. Once the injection is completed some injection pens have a visual or audible indication to confirm that the full dose has been delivered.

As used in this specification and the appended claims, the singular forms “a”, “an” and “the” include plural referents unless the content clearly dictates otherwise.

As used herein, the term “about” means ±10% of an associated numerical value.

Embodiments of the disclosure are now described with reference to the following examples.

### EXAMPLES

### Example 1

This Example follows a drug administration of subcutaneous delivery of 50 milligrams of study drug (long-acting GLP-1 receptor agonist of SEQ ID NO: 1) on Day 1, with the patient to be evaluated over a 48-hour period.

A 61-year-old female patient (7 years post-extensive bowel resection) presenting with severe malabsorption secondary to short bowel syndrome and a high output end jejunostomy was accepted for a clinical trial. Baseline stool output measurement was 22,508 milliliters for the 48 hours prior to the first dose of study drug. She was emptying her ostomy bag 8-10 times each day and several times at night. The patient had difficulty maintaining adequate hydration and nutrition with frequent modifications to her daily parenteral support (PS) and nutrition (total daily PS =2L). On admission to the study, the patient weighed 51.6 kg with a BMI of 19.4; baseline physical exam was unremarkable aside from ostomy and surgical scar; laboratory screening showed normal electrolytes, total protein and albumin with hemoglobin/hematocrit of 10.5/33.2.

On Day 1 of the study, the patient was administered (by subcutaneous injection) 50 milligrams of the study drug and followed for 48 hours. The patient's total stool output measurement in the 48 hours following the initial dose of study drug was 12,550 milliliters, reflecting a total stool output reduction of about 44% from baseline. Three days following the first dose of study drug, the patient's parenteral support was decreased by 500 milliliters per day (a 25% reduction) and was maintained at that level for the duration of the trial.

A second subcutaneous administration of study drug was given at 50 milligrams on Day 15. Within 48 hours of the second dose of study drug, the patient's total stool output was 13,950 milliliters, reflecting a 38% reduction from baseline. Two weeks following the second dose of study drug, the patient's body weight increased to 54.3 kg (from the 51.6 kg baseline). Overall, the patient remained improved for the duration of the 28 days clinical trial period.

In summary, this patient with longstanding SBS and a high output end jejunostomy demonstrated an immediate and remarkable clinical response to a dose of 50 mg subcutaneous injection of study drug.

### Example 2

Twelve patients were selected for evaluating the administration of a long-acting GLP-1 receptor agonist (i.e., SEQ ID NO: 1) at 50 mg or 100 mg subcutaneous (Q) weekly or biweekly dosing (Q2W), over a 4-week treatment regimen, and with a patient evaluation over a 6-week period. The placebo-controlled treatment arms and the patient phenotype are summarized in Tables 1 and 2, respectively.

TABLE 1
Dosing regimen for 4-week administration
with 6-week observation (PBO = placebo).

| | | Day | Day | Day | Day | Day |
|---|---|---|---|---|---|---|
| | | 0 | 7 | 14 | 21 | 28 |
| Group 1 | 50 mg | 50 mg | PBO | 50 mg | PBO | 50 mg |
| | Q2W | | | | | |
| Group 2 | 100 mg | 100 mg | PBO | 100 mg | PBO | 100 mg |
| | Q2W | | | | | |
| Group 3 | 50 mg | 50 mg | 50 mg | 50 mg | 50 mg | 50 mg |
| | weekly | | | | | |
| Group 4 | Placebo | PBO | PBO | PBO | PBO | PBO |

TABLE 2
Study demographics and patient phenotype.

| | | 50 mg | 50 mg | 100 mg |
|---|---|---|---|---|
| Demographics | Placebo | Q2W | weekly | Q2W |
| N (12)* | 2 | 3 | 3 | 4 |
| Sex (m/f) | 2(m) | 3(f) | 1(m); 2(f) | 3(m); 1(f) |
| Age (mean, yr.) | 56 | 50 | 58 | 47 |
| Weight (mean, kg.) | 69.4 | 66.5 | 67.4 | 70.9 |
| White | 2 | 2 | 2 | 4 |
| Black or | — | 1 | 1 | — |
| African American | | | | |
| Non-Hispanic | 2 | 3 | 3 | 3 |
| or Latino | | | | |

Post-Operative Phenotype

| | | | | |
|---|---|---|---|---|
| Ostomy | 2 | 1 | 3 | 2 |
| Colon-in- | — | 1 | 1 | 2 |
| Continuity (CIC) | | | | |

*Twelve patients were enrolled into the study; final efficacy results include 11 patients, with a safety population inclusive of 12 patients.

Typically, individuals with short bowel syndrome (SBS) exhibit impaired absorption with increased stool volume (diarrhea), resulting in a variety of health conditions, including dehydration, acute kidney failure, arrhythmia, weight loss, fatigue, and impaired wound healing. The total stool output (TSO), or stool volume, was measured for patients grouped into the dosing regimens (e.g., as detailed in Table 1), as a quantitative measure of dosing effectiveness. The TSO was measured for each treatment group along 24-hour periods and was recorded daily over the 6-week observational period, with the results summarized in Table 3. The results of Table 3 are shown graphically in FIG. 1.

TABLE 3
Weekly mean 24-hour TSO change from
baseline over the 6-week observational period.

| | | 50 mg | 50 mg | 100 mg | Any Dose |
|---|---|---|---|---|---|
| | Placebo | Q2W | weekly | Q2W | (n = 6) |
| TSO Mean Change | +32% | −30% | −8% | +16% | −37% |

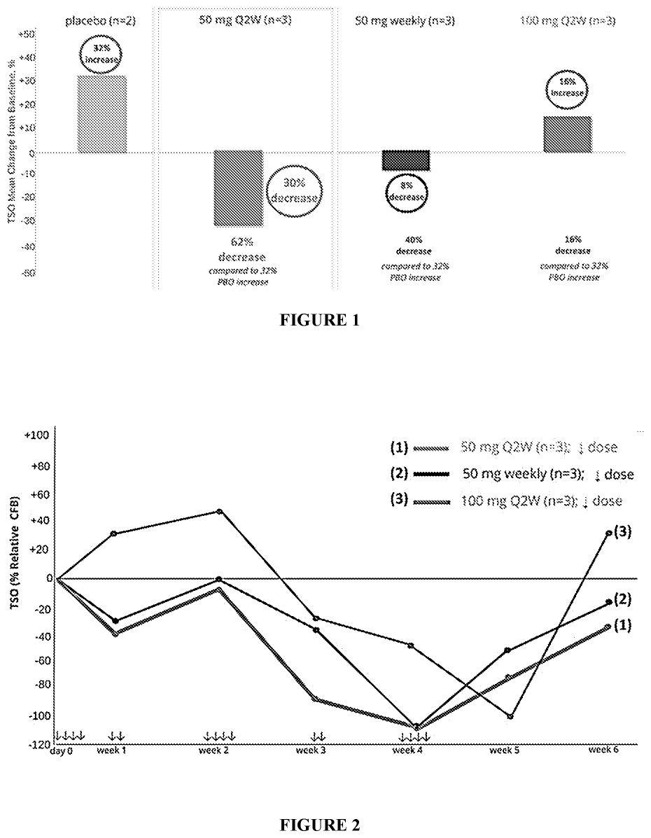

The results indicated that administration of the long-acting GLP-1 receptor agonist (SEQ ID NO: 1) acted as an “ileal brake,” where digestive motility is slowed in the gastrointestinal track allowing for more time for nutrient uptake. This slowing effect was observed as diminished stool output over the observational period. Administration was observed to result in a rapid onset with sustained TSO reduction in comparison to placebo. Six of 9 (67%) of patients provided the GLP-1 receptor agonist (“Any Dose,” Table 3) exhibited decreased TSO over the 6-week observational period, with a mean decrease of 37%. All 3 patients in the 50 mg Q2W group showed decreased TSO over the 6-week period, with this dosing regimen providing the greatest patient benefit, despite representing the lowest level of drug administration. The relative change in TSO from baseline (CFB) for each group receiving the study drug is illustrated in FIG. 2, with the overall benefit of the 50 mg Q2W dosing regimen indicating a 62% decrease in TSO relative to placebo (FIG. 1).

Adverse events were documented throughout the 6-week observational period. The results, as summarized in Table 4, indicated that there were no unexpected safety signals.

TABLE 4
Treatment-emergent adverse events summary.

| | | 50 mg | 50 mg | 100 mg |
|---|---|---|---|---|
| Demographics | Placebo | Q2W | weekly | Q2W |
| Nausea | 0 | 0 | 1 | 4* |
| Vomiting | 1 | 0 | 0 | 2* |
| Headache | 0 | 0 | 1 | 1 |
| Appetite Loss | 0 | 0 | 0 | 1 |
| Abdominal Pain | 0 | 1 | 0 | 0 |
| Hypertension | 1 | 0 | 0 | 0 |

*One patient was removed from the trial early due to nausea and vomiting in the 100 mg Q2W treatment group.

50 mg Q2W treatment demonstrated to be the most well-tolerated dosing/regimen. No treatment-related serious adverse events (SAEs) were observed, as well as no observed gastrointestinal stoma complications in any group. There were no instances of acceleration of neoplastic growth, intestinal obstruction, biliary and pancreatic disease, or fluid overload, including congestive heart failure.

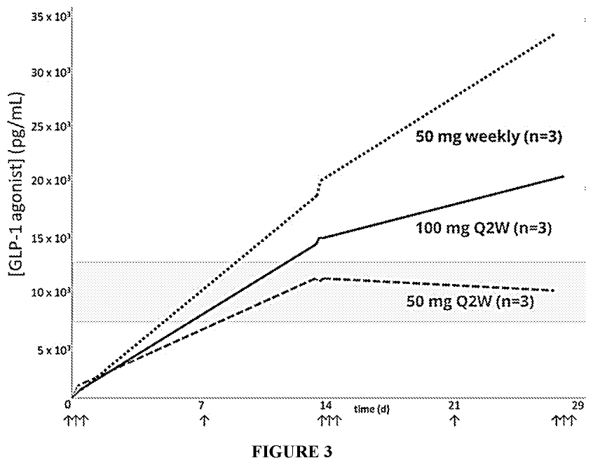

A pharmacokinetic (PK) analysis was performed, as illustrated in FIG. 3, which involved measuring GLP-1 receptor agonist concentration (in pg/mL) in patient samples throughout the 4-week dosing period. The 50 mg weekly and 100 mg Q2W treatment groups exhibited evidence of PK stacking effects, which indicated undesirable accumulation of the long-acting GLP-1 receptor agonist due to higher and/or more frequent dosing. The shaded region indicates the more favorable PK profile observed in the 50 Q2W treatment group samples.

There was an observed decrease in parenteral support (PS) requirements among patients receiving the GLP-1 agonist treatment. Parenteral nutritional support is typically provided as an ancillary treatment to patients who are unable to absorb adequate nutrients from the diet. As summarized in Table 5, there was a mean decrease in PS volume by week 2 of treatment, which was sustained over the 6-week observational period. Five of the 11 patients were on PS, each of which was placed into a GLP-1 receptor agonist treatment grouping. A mean decrease of 17% was observed by week 2 in this group, with a 28% mean decrease among the 3 of the 5 patients in this group which exhibited any decrease. Of these 3 patients, one exhibited chronic idiopathic constipation (CIC) and two were ostomy patients, none of which exhibited increased TSO.

TABLE 5
Parental support (PS) requirement among
GLP-1 receptor agonist-treated patients.

| | All PS | Patients with |
|---|---|---|
| | patients | PS decrease |
| | (n = 5) | (n =3) |
| Mean change in PS volume | −17% | −28% |
| requirement from Baseline | | |

In summary, the GLP-1 receptor agonist (SEQ ID NO: 1) demonstrated an effective reduction in the mean 24-hour total stool output (TSO), with the 50 mg Q2W dosing treatment arm exhibiting the greatest effect, a 30% reduction at week six versus a 32% increase in the placebo arm — an overall mean relative reduction of 62%. The GLP-1 receptor agonist demonstrated effective reduction in PS volume, with a mean decrease of 17% among the 5 patients under PS by week 2, lasting throughout the 6-week observational period. The 50 mg Q2W treatment arm demonstrated the most favorable PK profile of the treatment arms tested. Overall, 50 mg Q2W treatment exhibited the highest efficacy (despite representing the lowest level of drug administration), most favorable safety, tolerability, and PK profile.

SEQUENCES
Exenatide fused to AE864XTEN
SEQ ID NO: 1
HGEGTFTSDLSKQMEEEAVRLFIEWLKNGGPSSGAPPPSGGSPAGSPTST
EEGTSESATPESGPGTSTEPSEGSAPGSPAGSPTSTEEGTSTEPSEGSAP
GTSTEPSEGSAPGTSESATPESGPGSEPATSGSETPGSEPATSGSETPGS
PAGSPTSTEEGTSESATPESGPGTSTEPSEGSAPGTSTEPSEGSAPGSPA
GSPTSTEEGTSTEPSEGSAPGTSTEPSEGSAPGTSESATPESGPGTSTEP
SEGSAPGTSESATPESGPGSEPATSGSETPGTSTEPSEGSAPGTSTEPSE
GSAPGTSESATPESGPGTSESATPESGPGSPAGSPTSTEEGTSESATPES
GPGSEPATSGSETPGTSESATPESGPGTSTEPSEGSAPGTSTEPSEGSAP
GTSTEPSEGSAPGTSTEPSEGSAPGTSTEPSEGSAPGTSTEPSEGSAPGS
PAGSPTSTEEGTSTEPSEGSAPGTSESATPESGPGSEPATSGSETPGTSE
SATPESGPGSEPATSGSETPGTSESATPESGPGTSTEPSEGSAPGTSESA
TPESGPGSPAGSPTSTEEGSPAGSPTSTEEGSPAGSPTSTEEGTSESATP
ESGPGTSTEPSEGSAPGTSESATPESGPGSEPATSGSETPGTSESATPES
GPGSEPATSGSETPGTSESATPESGPGTSTEPSEGSAPGSPAGSPTSTEE
GTSESATPESGPGSEPATSGSETPGTSESATPESGPGSPAGSPTSTEEGS
PAGSPTSTEEGTSTEPSEGSAPGTSESATPESGPGTSESATPESGPGTSE
SATPESGPGSEPATSGSETPGSEPATSGSETPGSPAGSPTSTEEGTSTEP
SEGSAPGTSTEPSEGSAPGSEPATSGSETPGTSESATPESGPGTSTEPSE
GSAPG

What is claimed is:

1. A method for treating short bowel syndrome and/or high output ostomy, the method comprising administering to a patient in need thereof a long-acting GLP-1 receptor agonist.

2. The method of claim 1, wherein the patient has a high output ostomy.

3. The method of claim 1 or 2, wherein the patient has an end jejunostomy.

4. The method of any one of claims 1 to 3, wherein the long-acting GLP-1 agonist is an exenatide or GLP-1 fusion with a half-life extension polypeptide.

5. The method of claim 4, wherein the half-life extension polypeptide is fused to the C-terminus of the exenatide or GLP-1.

6. The method of claim 4 or 5, wherein the half-life extension polypeptide comprises an albumin amino acid sequence, a transferrin amino acid sequence, elastin-like peptide amino acid sequence, or is an unstructured polypeptide.

7. The method of claim 6, wherein administration of the GLP-1 receptor agonist composition results in an effective amount of the GLP-1 receptor agonist in circulation for at least about one week upon subcutaneous injection.

8. The method of claim 7, wherein the long-acting GLP-1 receptor agonist is administered by subcutaneous injection.

9. The method of any one of claims 6 to 8, wherein the long-acting GLP-1 receptor agonist comprises the amino acid sequence of SEQ ID NO: 1.

10. The method of claim 9, wherein the long-acting GLP-1 receptor agonist is administered at a unit dose of about 12 milligrams to about 250 milligrams.

11. The method of claim 10, wherein the unit dose is about 50 mg to about 150 mg.

12. The method of claim 10, wherein the unit dose is about 50 mg.

13. The method of claim 10, wherein the unit dose is about 100 mg.

14. The method of claim 10, wherein the unit dose is about 150 mg.

15. The method of any one of claims 1 to 14, wherein the long-acting GLP-1 receptor agonist is administered about once weekly.

16. The method of any one of claims 1 to 14, wherein the long-acting GLP-1 receptor agonist is administered about twice monthly.

17. The method of any one of claims 1 to 14, wherein the long-acting GLP-1 receptor agonist is administered about once monthly.

18. The method of any one of claims 1 to 17, wherein the patient exhibits an average daily reduction of ostomy output of at least about 10% from baseline, or at least about 20% from baseline.

19. The method of claim 18, wherein parenteral support is reduced by at least about 10% from baseline, or at least about 20% from baseline, or at least about 30% from baseline.

20. The method of any one of claims 1 to 19, wherein the patient does not experience substantial adverse effects, such as symptoms of nausea, vomiting, hypoglycemia, pancreatitis, liver injury, and/or kidney function.

21. A method for treating a patient having short bowel syndrome, the method comprising administering to the patient by subcutaneous injection a unit dose of from about 12.5 mg to about 200 mg of the long-acting GLP-1 receptor agonist of SEQ ID NO: 1, the administration being from about once weekly to about once monthly.

22. The method of claim 21, wherein the patient has an end jejunostomy.

23. The method of claim 21, wherein the patient has a high output ostomy.

24. The method of any one of claims 21 to 23, wherein subcutaneous administration of the GLP-1 receptor agonist unit dose results in an effective amount of the GLP-1 receptor agonist in circulation for at least about one week.

25. The method of any one of claims 21 to 24, wherein the unit dose is about 50 mg to about 150 mg.

26. The method of claim 25, wherein the unit dose is about 50 mg.

27. The method of claim 25, wherein the unit dose is about 100 mg.

28. The method of claim 25, wherein the unit dose is about 150 mg.

29. The method of any one of claims 21 to 28, wherein the long-acting GLP-1 receptor agonist is administered about once weekly.

30. The method of any one of claims 21 to 28, wherein the long-acting GLP-1 receptor agonist is administered about twice monthly.

31. The method of any one of claims 21 to 28, wherein the long-acting GLP-1 receptor agonist is administered about once monthly.

32. The method of any one of claims 21 to 31, wherein the patient does not experience substantial adverse effects, such as symptoms of nausea, vomiting, hypoglycemia, pancreatitis, liver injury, and/or kidney function.

33. A unit dose composition of a long-acting GLP-1 receptor agonist defined by SEQ ID NO: 1, the unit dose composition comprising from about 12.5 mg to about 200 mg of the long acting GLP-1 receptor agonist formulated for subcutaneous administration.

34. The unit dose composition of claim 33, wherein the unit dose is about 50 mg.

35. The unit dose composition of claim 33, wherein the unit dose is about 100 mg.

36. The unit dose composition of claim 33, wherein the unit dose is about 150 mg.

37. The unit dose composition of any one of claims 33 to 36, wherein the unit dose is contained in an injection pen.

38. The unit dose composition of claim 37, wherein the injection pen contains at least four unit doses.

39. The unit dose composition of any one of claims 33 to 38, wherein the unit doses are no more than about 1 mL or no more than about 2 mLs in volume.
